(19) United States
(12) Patent Application Publication
(10) Pub. No.: US 2016/0272697 A2
(43) Pub. Date: Sep. 22, 2016

(54) Neutralizing Antibodies to Nipah and Hendra Virus

(71) Applicant: The Henry M. Jackson Foundation for the Advancement of Military Medicine, Inc., Bethesda, MD (US)

(72) Inventors: Christopher C. Broder, Silver Spring, MD (US); Deborah L. Fusco, Silver Spring, MD (US); Kai Xu, New York, NY (US); Dimitar B. Nikolov, New York, NY (US)

(21) Appl. No.: 14/114,465

(22) PCT Filed: Apr. 30, 2012
(86) PCT No.: PCT/US2012/035806

§ 371 Date: Oct. 28, 2013

Related U.S. Application Data
(60) Provisional application No. 61/480,151, filed on Apr. 28, 2011.

(51) Int. Cl. C07K 16/10

(52) U.S. Cl. CPC C07K 16/1027

(57) ABSTRACT

The invention described herein provides novel peptides. The novel peptides are useful alone or as portions of larger molecules, such as antibodies or antibody fragments, that can be used to treat or prevent infection of Nipah virus and/or Hendra virus.

### CROSS-REFERENCE TO RELATED APPLICATIONS

The application claims priority to U.S. Provisional Application No. 61/480,151 filed 28 Apr. 2011, which is incorporated by reference.

### STATEMENT REGARDING FEDERALLY SPONSORED RESEARCH OR DEVELOPMENT

Part of the work performed during development of this invention utilized U.S. Government funds under National Institutes of Health Grant No. U01A1077995. The U.S. Government has certain rights in this invention.

### REFERENCE TO SEQUENCE LISTING

A computer readable text file, entitled “044508-5036-SequenceListing.txt,” created on or about 28 Oct. 2013 with a file size of about 126 kb contains the sequence listing for this application and is hereby incorporated by reference in its entirety.

### BACKGROUND OF THE INVENTION

1. Field of the Invention

The invention described herein provides novel peptides. The novel peptides are useful alone or as portions of larger molecules, such as antibodies or antibody fragments, that can be used to treat or prevent infection of Nipah virus and/or Hendra virus.

2. Background of the Invention

Nipah virus (NiV) and Hendra virus (HeV) are closely related emerging paramyxoviruses that comprise the Henipavirus genus. Paramyxoviruses are negative-sense RNA containing enveloped viruses and contain two major membrane-anchored envelope glycoproteins that are required for infection of a receptive host cell. All members contain an F glycoprotein which mediates pH-independent membrane fusion between the virus and its host cell, while the second attachment glycoprotein can be either a hemagglutinin-neuraminidase protein (HN), a hemagglutinin protein (H), or a G protein depending on the particular virus (reviewed in Lamb, R. A. and Kolakofsky, D. 2001 in Fields Virology, eds. Knippe, D. M. & Howley, P. M., Lippincott Williams & Wilkins, Philadelphia, pp. 1305-1340). As with all paramyxoviruses, these glycoproteins are also the principal antigens to which virtually all neutralizing antibodies are directed. A number of studies have shown the importance of neutralizing antibodies in recovery and protection from viral infections (Dimitrov, D. S. 2004 Nat Rev Microbiol 2:109-122).

The broad species tropisms and the ability to cause fatal disease in both animals and humans distinguish HeV and NiV from all other known paramyxoviruses (reviewed in Eaton, B. T., Microbes Infect., 3:277-278 (2001)). They are Biological Safety Level-4 (BSL-4) pathogens, and are on the NIAID Biodefense research agenda as zoonotic emerging category C priority pathogens that could be used as bioterror agents. The henipaviruses can be amplified and cause disease in large animals and be aerosol transmitted to humans where disease can be a severe respiratory illness and febrile encephalitis. They can be readily grown in cell culture or embryonated chicken eggs, produce high un-concentrated titers (˜10⁸ TCID₅₀/ml; Crameri, G., et al. J Virol. Methods, 99:41-51 (2002)), and are highly infectious (Field, H., et al. Microbes Infect., 3:307-314 (2001); Hooper, P., et al. Microbes Infect., 3:315-322 (2001)).

NiV has re-emerged in Bangladesh. Several important observations in these most recent outbreaks have been made, including a higher incidence of acute respiratory distress syndrome, person-to-person transmission, and significantly higher case fatality rates (60-100%) than in Malaysia (about 40%) where the virus was discovered or suspected to have originated (Anonymous Wkly Epidemiol Rec 79:168-171 (2004); Anonymous Health and Science Bulletin (ICDDR,B) 2:5-9 (2004); Butler, D., Nature 429:7 (2004); Enserink, M., Science 303:1121 (2004); Hsu, V. P., et al. Emerg. Infect. Dis., 10:2082-2087 (2004)). Currently, there are no therapeutics for NiV or HeV-infected individuals, and a vaccine for prevention of disease in human or livestock populations does not exist. Although antibody responses were detected in infections caused by these viruses, human monoclonal antibodies (hmabs) have not been identified against either virus. Therefore, the development of neutralizing hmAbs against NiV and HeV could have important implications for prophylaxis and passive immunotherapy. In addition, the characterization of the epitopes of the neutralizing antibodies could provide helpful information for development of candidate vaccines and drugs. Finally, such antibodies could be used for diagnosis and as research reagents.

### SUMMARY OF THE INVENTION

The present invention is directed to novel peptides, antibodies and antibody fragments that bind Hendra virus and/or Nipah virus.

The present invention is also directed to methods of using the novel peptides, antibodies and antibody fragments, such methods of treatment, methods of prevention and diagnostic methods.

The present invention also relates to nucleic acids encoding the novel peptides, antibodies and antibody fragments of the present invention, including vectors and host cells containing the nucleic acids.

### BRIEF DESCRIPTION OF THE DRAWINGS

FIG. 1 depicts the non-linear epitope of the Hendra and Nipha virus to which the antibodies of the present invention will bind.

FIG. 2 depicts the ability of some novel peptides of the present invention to bind Hendra virus soluble G protein (HeV-sG). Briefly, HeV-sG was coated overnight on a 96-well ELISA plate. The next day the plate was blocked and washed prior to the addition of various concentrations of select novel peptides. The plate was incubated for one hour at room temperature followed by washing. An HRP-conjugated secondary antibody that binds to the novel peptides was added and the plate was incubated for another hour at room temperature. The plate was washed and the substrate was added. After a thirty minute incubation at room temperature, the plate was read at 405 nm. As can be seen, a majority of the novel peptides tested were able to bind HeV-sG with two variants (Peptide of SEQ ID NO:2 and SEQ ID NO:6) binding similarly. To test the binding of the peptides to mutants of HeV-sG, this assay can be repeated, but the plate would be coated with the mutant versions of HeV-sG instead of native HeV-sG.

FIG. 3 depicts the ability of some of the novel peptides of the present invention to inhibit the interaction between HeV-sG and Ephrin-B2. Ephrin-B2 was coated overnight on a 96-well ELISA plate, and the subsequent day the plate was blocked and washed. A premixed solution containing a constant concentration of HeV-sG with various concentrations of novel peptides was added to the plate and incubated at room temperature for one hour. The plate was then washed prior to the addition of an HRP-conjugated secondary antibody that binds HeV-sG. The plate was incubated for an additional hour at room temperature, washed and substrate was added. Following a thirty minute incubation at room temperature, the plate was read at 405 nm. The novel peptides displayed a range of ability to prevent interaction between Ephrin-B2 and HeV-sG. For example, two variants (peptides of SEQ ID NO:2 and SEQ ID NO:6) had similar levels of interaction. This competition assay can also be used to determine the ability of the novel peptides of the present invention to inhibit the interaction between receptor and mutants of HeV-sG by replacing native HeV-sG with the mutant versions in the pre-mixed solution of G and novel peptides.

### DETAILED DESCRIPTION OF THE INVENTION

The present invention is directed to novel peptides. The terms “peptide,” “polypeptide” and “protein” are used interchangeably herein. In particular, the present invention provides for peptides comprising amino acid sequences at least 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical to the amino acid sequence of SEQ ID NO: 2, except that the novel peptides do not consist of or comprise an amino acid sequence that is 100% identical to the amino acid sequence of SEQ ID NO: 1.

The amino acid sequence of SEQ ID NO:1 as disclosed herein is the variable heavy chain from a series of antibodies disclosed in U.S. Pat. No. 7,988,971, also published as WO2006/137931, both of which are incorporated by reference. In particular, the amino acid sequence of SEQ ID NO: 1, which is disclosed below, is the amino acid sequence of the variable heavy chain for the m102 series of antibodies disclosed in the '971 U.S. patent.

(SEQ ID NO: 1)
EVQVIQSGADVKKPGSSVKVSCKSSGGTFSKYAINWVRQA
PGQGLEWMGGIIPILGIANYAQKFQGRVTITTDESTSTAY
MELSSLRSEDTAVYYCARGWGREQLAPHPSQYYYYYYGMD
VWGQGTTVTVSS

The amino acid sequence of SEQ ID NO: 2 is disclosed below.

(SEQ ID NO: 2)
GWGREQFAPHPSQYYYYYYGMDV

In other embodiments, the present invention provides for peptides that consist essentially of, or consist of an amino acid sequence at least 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical to the amino acid sequence of SEQ ID NO: 2, except that the novel peptides do not consist of an amino acid sequence that is 100% identical to the amino acid sequence of SEQ ID NO: 1. In another embodiment, the novel peptides of the present invention do not consist of any amino acid sequences that are 100% identical to the amino acid sequences of SEQ ID NOs: 1 and 32-383 disclosed herein.

In certain select embodiments of the present invention, the peptides of the present invention comprise, consist essentially of, or consist of an amino acid sequence that includes but is not limited to the amino acid sequence of SEQ ID NO: 2, the amino acid sequence of SEQ ID NO: 3, the amino acid sequence of SEQ ID NO: 4, the amino acid sequence of SEQ ID NO: 5, the amino acid sequence of SEQ ID NO: 6, the amino acid sequence of SEQ ID NO: 7, the amino acid sequence of SEQ ID NO: 8, the amino acid sequence of SEQ ID NO: 9, the amino acid sequence of SEQ ID NO: 10, the amino acid sequence of SEQ ID NO: 11, the amino acid sequence of SEQ ID NO: 12, the amino acid sequence of SEQ ID NO: 13, the amino acid sequence of SEQ ID NO: 14, the amino acid sequence of SEQ ID NO: 15, the amino acid sequence of SEQ ID NO: 16, the amino acid sequence of SEQ ID NO: 17, the amino acid sequence of SEQ ID NO: 18, the amino acid sequence of SEQ ID NO: 19, the amino acid sequence of SEQ ID NO: 20, the amino acid sequence of SEQ ID NO: 21, the amino acid sequence of SEQ ID NO: 22, the amino acid sequence of SEQ ID NO: 23, the amino acid sequence of SEQ ID NO: 384, the amino acid sequence of SEQ ID NO: 385, and the amino acid sequence of SEQ ID NO: 386, except that the novel peptides do not have an amino acid sequence that is 100% identical to the amino acid sequence of SEQ ID NO:1 disclosed herein. In another embodiment, the novel peptides of the present invention do not consist of any of amino acid sequences that are 100% identical to the amino acid sequences of SEQ ID NOs: 1 and 32-383 disclosed herein.

TABLE I

| | # of | Sequence |
|---|---|---|
| SEQ ID NO: | Amino | |
| (description) | Acids | |
| 2 | 23 | GWGREQFAPHPSQYYYYYYGMDV |
| 3 | 23 | GWGREQDAPHPSQYYYYYYGMDV |
| 4 | 23 | GWGREQAAPHPSQYYYYYYGMDV |
| 5 | 23 | GWGREQLAAHPSQYYYYYYGMDV |
| 6 | 23 | GWGREQLAPAPSQYYYYYYGMDV |
| 7 | 23 | GWGREQLAPNPSQYYYYYYGMDV |
| 8 | 23 | GWGREQYAPHPSQYYYYYYGMDV |
| 9 | 23 | GWGREQLAPHLSQYYYYYYGMDV |
| 10 | 23 | GWGREQFAPHLSQYYYYYYGMDV |
| 11 | 23 | GWGREQFAPHLWQYYYYYYGMDV |
| 12 | 23 | GWGREQFAPNLWQYYYYYYGMDV |
| 13 | 23 | GWGREQFSPNPWQYYYYYYGMDV |
| 14 | 23 | GWGREQFSPNLWQYYYYYYGMDV |
| 15 | 23 | GWGREQLAPHLWQYYYYYYGMDV |
| 16 | 23 | GWGREQLAPNLWQYYYYYYGMDV |
| 17 | 23 | GWGREQLAPAPWQYYYYYYGMDV |
| 18 | 23 | GWGREQFAAHPSQYYYYYYGMDV |
| 19 | 23 | GWGREQFAPAPSQYYYYYYGMDV |
| 20 | 23 | GWGREQLAAAPSQYYYYYYGMDV |
| 21 | 23 | GWGREQYAPAPSQYYYYYYGMDV |
| 22 | 23 | GWGREQYAAHPSQYYYYYYGMDV |
| 23 | 23 | GWGREQYAPHLSQYYYYYYGMDV |
| 24 (generic) | 23 | GWGREQX₁X₂X₃X₄X₅X₆QYYYYYYGMDV |
| 25 | 8 | GGTFSNYA |
| 26 | 7 | IPILGIA |
| 27 | 7 | QSVRNNY |
| 28 | 3 | NGS |
| 29 | 10 | QQYGNSRRVT |

In additional embodiments, the novel peptides comprise, consist essentially of or consist of amino acid sequences that are not 100% identical to any of the variable heavy or variable light chain amino acid sequences that are disclosed U.S. Pat. No. 7,988,971. In particular, the novel peptides of the present invention do not consist of any of the amino acid sequences of SEQ ID NOs: 1 and 32-383 disclosed herein, which are also disclosed as SEQ ID NOs: 1-416 in the '971 patent (WO2006/137931).

As disclosed herein, the novel peptides of the present invention comprising amino acid sequences of SEQ ID NOs: 2-23 and 384-386 are each useful as a complementarity determining region (CDR) of an antibody or antibody fragment that binds to Hendra virus and/or Nipah virus. In one embodiment, the novel peptides with amino acid sequences of any one of SEQ ID NOs: 2-23 and 384-386 of the present invention are, alone, considered to be an antibody fragment that could be useful in binding Hendra and/or Nipah virus. The generic sequence amino acid of SEQ ID NO: 24 above indicates just one region where certain residues within the novel peptides of the present invention may be present within an antibody or antibody fragment and may vary according to the parameters of the present invention and still retain the ability to bind Hendra virus and/or Nipah virus.

For example, any of residues X₁₋₆ of SEQ ID NO: 24 can be present or absent and can be any single amino acid, provided that the amino acid sequence is not the amino acid sequence of SEQ ID NO:1. In select embodiments of the present invention, residue X₁ can be lysine (L), phenylalanine (F), alanine (A), tyrosine (Y) or aspartic acid (D). In additional select embodiments of the present invention, residue X₂ can be alanine (A) or serine (S). In additional select embodiments of the present invention, residue X₃ alanine (A) or proline (P). In additional select embodiments of the present invention, residue X₄ can be histidine (H), alanine (A) or asparagine (N). In additional select embodiments of the present invention, residue X₅ can be proline (P) or lysine (L). In additional select embodiments of the present invention, residue X₆ can be serine (S) or tryptophan (W).

The novel peptides of the present invention can serve as at least one CDR of an antibody or antibody fragment that can bind to a specific epitope present on Hendra virus and/or Nipah virus. The antibodies of the present invention can be monoclonal or polyclonal. As used herein, the term “antibody” means an immunoglobulin molecule or a fragment of an immunoglobulin molecule having the ability to specifically bind to a particular antigen. Antibodies are well known to those of ordinary skill in the science of immunology. As used herein, the term antibody includes fragments of full-length antibodies that specifically bind one or more antigens. Such fragments are also well known in the art and are regularly employed both in vitro and in vivo. Examples of fragments of full length antibodies that are encompassed by the term antibody include but are not limited to F(ab′)2, Fab, Fv, Fd fragments, as well as scFv peptides and the like.

In addition to Fabs, smaller antibody fragments and epitope-binding peptides, including the novel peptides of the present invention, that have binding specificity for the epitopes defined by the Hendra and Nipah antibodies are also contemplated by the present invention and can also be used to bind or neutralize the virus. For example, single chain antibodies can be constructed according to the method of U.S. Pat. No. 4,946,778, which is incorporated by reference. Single chain antibodies comprise the variable regions of the light and heavy chains joined by a flexible linker moiety. Another smaller antibody fragment that the invention provides is the antibody fragment known as the single domain antibody or Fd, which comprises an isolated variable heavy chain domain. Techniques for obtaining a single domain antibody with at least some of the binding specificity of the full-length antibody from which they are derived are known in the art.

In one specific embodiment, the novel peptides of the present invention serve as the CDR1 portion of the heavy chain of an antibody or antibody fragment. In another specific embodiment, the novel peptides of the present invention serve as the CDR2 portion of the heavy chain of an antibody or antibody fragment. In another specific embodiment, the novel peptides of the present invention serve as the CDR3 portion of the heavy chain of an antibody or antibody fragment. In another specific embodiment, the novel peptides of the present invention serve as the CDR1 portion of the light chain of an antibody or antibody fragment. In another specific embodiment, the novel peptides of the present invention serve as the CDR2 portion of the light chain of an antibody or antibody fragment. In another specific embodiment, the novel peptides of the present invention serve as the CDR3 portion of the light chain of an antibody or antibody fragment.

In one embodiment, any of the novel peptides described can serve as a heavy chain CDR3 for an antibody or antibody fragment, with the antibody or antibody fragment further comprising at least one additional heavy chain CDR. In a more specific embodiment, any of the novel peptides described can serve as a heavy chain CDR3 for an antibody or antibody fragment, and a peptide comprising the amino acid sequence of SEQ ID NO: 25 or SEQ ID NO: 26 can serve as an additional heavy chain CDR, for example either CDR1 or CDR2. In another embodiment, any of the novel peptides described can serve as a heavy chain CDR3 for an antibody or antibody fragment, with the antibody or antibody fragment further comprising at least two additional heavy chain CDRs. In another specific embodiment, any of the novel peptides described can serve as a heavy chain CDR3 for an antibody or antibody fragment, and peptides comprising the amino acid sequences of SEQ ID NO: 25 and SEQ ID NO: 26 can each serve as two additional heavy chain CDRs, for example CDR1 and CDR2, or vice versa.

In additional embodiments, any of the novel peptides described can serve as a heavy chain CDR3 for an antibody or antibody fragment, with the antibody or antibody fragment further comprising at least one light chain CDR, and a peptide comprising the amino acid sequence of SEQ ID NO: 27, SEQ ID NO:28 or SEQ ID NO: 29 can serve as either light chain CDR1, CDR2 or CDR3. In another embodiment, any of the novel peptides described can serve as a heavy chain CDR3 for an antibody or antibody fragment, with the antibody or antibody fragment further comprising at least two additional light chain CDRs. In another specific embodiment, any of the novel peptides described can serve as a heavy chain CDR3 for an antibody or antibody fragment, and peptides comprising the amino acid sequences of SEQ ID NO: 27, SEQ ID NO:28 or SEQ ID NO: 29 can serve as two additional light chain CDRs, for example light chain CDR1, CDR2 or CDR3. In particular, a peptide with the amino acid sequence of SEQ ID NO:27 can serve as the light chain CDR1 and a peptide with an amino acid sequence of SEQ ID NO:28 or SEQ ID NO:29 can interchangeably serve as the light chain CDR2 or CDR3. In another specific embodiment, any of the novel peptides described can serve as a heavy chain CDR3 for an antibody or antibody fragment, with the antibody or antibody fragment further comprising at least three additional light chain CDRs. In another specific embodiment, any of the novel peptides described can serve as a heavy chain CDR3 for an antibody or antibody fragment, and peptides comprising the amino acid sequences of SEQ ID NO: 27, SEQ ID NO:28 or SEQ ID NO: 29 can serve as three additional light chain CDRs, for example light chain CDR1, CDR2 and CDR3. In particular, a peptide with the amino acid sequence of SEQ ID NO:27 can serve as the light chain CDR1 and a peptide with an amino acid sequence of SEQ ID NO:28 can serve as the light chain CDR2 and a peptide with an amino acid sequence of SEQ ID NO:29 can serve as the light chain CDR3.

In additional embodiments, any of the novel peptides described can serve as a heavy chain CDR3 for an antibody or antibody fragment, with the antibody or antibody fragment further comprising at least one, two, three, four or five additional CDRs. In specific embodiments, any of the novel peptides described can serve as a heavy chain CDR for an antibody or antibody fragment, with the antibody or antibody fragment further comprising at least two additional CDRs. In another specific embodiment, any of the novel peptides described can serve as a heavy chain CDR for an antibody or antibody fragment, and peptides comprising the amino acid sequences of SEQ ID NO:25, SEQ ID NO:26, SEQ ID NO: 27, SEQ ID NO:28 or SEQ ID NO: 29 can serve as at least one, two, three, four or five additional CDR(s). In particular, any of the novel peptides described can serve as a heavy chain CDR for an antibody or antibody fragment, and a peptide comprising the amino acid sequences of SEQ ID NO: 25 can serve as a heavy chain CDR1, a peptide comprising the amino acid sequence of SEQ ID NO: 26 can serve as a heavy chain CDR2, a peptide with the amino acid sequence of SEQ ID NO:27 can serve as a light chain CDR1, a peptide with an amino acid sequence of SEQ ID NO:28 can serve as a light chain CDR2, and/or a peptide with an amino acid sequence of SEQ ID NO:29 can serve as a light chain CDR3.

Additional embodiments are included in the table below. In these embodiments in Table II, the antibodies or antibody fragments comprises at least a peptide with an amino acid sequence of fragment (6) below and may further comprise one or more of enumerated fragments 1-5.

TABLE II

| | (1) V_{L}- | (2) V_{L}- | (3) V_{L}- | (4) V_{H}- | (5) V_{H}- | (6) V_{H}- |
|---|---|---|---|---|---|---|
| | CDR1 | CDR2 | CDR3 | CDR1 | CDR2 | CDR3 |
| | SEQ | SEQ | SEQ | SEQ | SEQ | Any of |
| | ID NO: | ID NO: | ID NO: | ID NO: | ID NO: | SEQ ID NOs: |
| 202 | 314 | 316 | 318 | 306 | 308 | 2-24; 384-386 |
| series | | | | | | |
| 203 | 322 | 324 | 326 | 306 | 308 | 2-24; 384-386 |
| series | | | | | | |
| 204 | 330 | 332 | 334 | 306 | 308 | 2-24; 384-386 |
| series | | | | | | |
| 205 | 338 | 340 | 342 | 306 | 308 | 2-24; 384-386 |
| series | | | | | | |
| 211 | 346 | 348 | 350 | 306 | 308 | 2-24; 384-386 |
| series | | | | | | |
| 212 | 354 | 356 | 358 | 306 | 308 | 2-24; 384-386 |
| series | | | | | | |
| 213 | 362 | 364 | 366 | 306 | 308 | 2-24; 384-386 |
| series | | | | | | |
| 215 | 370 | 372 | 374 | 306 | 308 | 2-24; 384-386 |
| series | | | | | | |
| 216 | 378 | 380 | 382 | 306 | 308 | 2-24; 384-386 |
| series | | | | | | |

Any of the series of antibodies or antibody fragments in Table II above may or may not include one or more framework regions as well. Amino acid sequences of framework regions are enumerated in the sequence listing disclosed herein. In specific embodiments, the antibody series in Table II above may or may not have from one to six of framework regions (FRs) A-H from Tale III below. In general, FRs A-D are framework regions for heavy chain portions of an antibody or antibody fragment and FRs E-H are framework regions for light chain portions of an antibody or antibody fragment.

TABLE III

| Ab | (A) FR1 | (B) FR2 | (C) FR3 | (D) FR4 | (E) FR5 | (F) FR6 | (G) FR7 | (H) FR8 |
|---|---|---|---|---|---|---|---|---|
| series | SEQ ID: | SEQ ID: | SEQ ID: | SEQ ID: | SEQ ID: | SEQ ID: | SEQ ID: | SEQ ID: |
| 202 | 305 | 307 | 309 | 311 | 313 | 315 | 317 | 319 |
| 203 | 305 | 307 | 309 | 311 | 321 | 323 | 325 | 327 |
| 204 | 305 | 307 | 309 | 311 | 329 | 331 | 333 | 335 |
| 205 | 305 | 307 | 309 | 311 | 337 | 339 | 341 | 343 |
| 211 | 305 | 307 | 309 | 311 | 345 | 347 | 349 | 351 |
| 212 | 305 | 307 | 309 | 311 | 353 | 355 | 357 | 359 |
| 213 | 305 | 307 | 309 | 311 | 361 | 363 | 365 | 367 |
| 215 | 305 | 307 | 309 | 311 | 369 | 371 | 373 | 375 |
| 216 | 305 | 307 | 309 | 311 | 377 | 379 | 381 | 383 |

Accordingly, the present invention provides for novel antibodies or antibody fragments that bind to a specific epitope present on Hendra virus and/or Nipah virus, provided the antibodies or antibody fragments do not comprise (1) a heavy chain variable region with an amino acid sequence of SEQ ID NO: 32 and a light chain variable region with an amino acid sequence of SEQ ID NO: 40 as disclosed herein, (2) a heavy chain variable region with an amino acid sequence of SEQ ID NO: 48 and a light chain variable region with an amino acid sequence of SEQ ID NO: 56 as disclosed herein, (3) a heavy chain variable region with an amino acid sequence of SEQ ID NO: 64 and a light chain variable region with an amino acid sequence of SEQ ID NO: 72 as disclosed herein, (4) a heavy chain variable region with an amino acid sequence of SEQ ID NO: 80 and a light chain variable region with an amino acid sequence of SEQ ID NO: 88 as disclosed herein, (5) a heavy chain variable region with an amino acid sequence of SEQ ID NO: 96 and a light chain variable region with an amino acid sequence of SEQ ID NO: 104 as disclosed herein, (6) a heavy chain variable region with an amino acid sequence of SEQ ID NO: 112 and a light chain variable region with an amino acid sequence of SEQ ID NO: 120 as disclosed herein, (7) a heavy chain variable region with an amino acid sequence of SEQ ID NO: 128 and a light chain variable region with an amino acid sequence of SEQ ID NO: 136 as disclosed herein, (8) a heavy chain variable region with an amino acid sequence of SEQ ID NO: 144 and a light chain variable region with an amino acid sequence of SEQ ID NO: 152 as disclosed herein, (9) a heavy chain variable region with an amino acid sequence of SEQ ID NO: 160 and a light chain variable region with an amino acid sequence of SEQ ID NO: 168 as disclosed herein, (10) a heavy chain variable region with an amino acid sequence of SEQ ID NO: 176 and a light chain variable region with an amino acid sequence of SEQ ID NO: 184 as disclosed herein, (11) a heavy chain variable region with an amino acid sequence of SEQ ID NO: 192 and a light chain variable region with an amino acid sequence of SEQ ID NO: 200 as disclosed herein, (12) a heavy chain variable region with an amino acid sequence of SEQ ID NO: 208 and a light chain variable region with an amino acid sequence of SEQ ID NO: 216 as disclosed herein, (13) a heavy chain variable region with an amino acid sequence of SEQ ID NO: 224 and a light chain variable region with an amino acid sequence of SEQ ID NO: 232 as disclosed herein, (14) a heavy chain variable region with an amino acid sequence of SEQ ID NO: 240 and a light chain variable region with an amino acid sequence of SEQ ID NO: 248 as disclosed herein, (15) a heavy chain variable region with an amino acid sequence of SEQ ID NO: 256 and a light chain variable region with an amino acid sequence of SEQ ID NO: 264 as disclosed herein, (16) a heavy chain variable region with an amino acid sequence of SEQ ID NO: 272 and a light chain variable region with an amino acid sequence of SEQ ID NO: 280 as disclosed herein, (17) a heavy chain variable region with an amino acid sequence of SEQ ID NO: 288 and a light chain variable region with an amino acid sequence of SEQ ID NO: 296 as disclosed herein, (18) a heavy chain variable region with an amino acid sequence of SEQ ID NO: 304 and a light chain variable region with an amino acid sequence of SEQ ID NO: 312 as disclosed herein, (19) a heavy chain variable region with an amino acid sequence of SEQ ID NO: 304 and a light chain variable region with an amino acid sequence of SEQ ID NO: 320 as disclosed herein, (20) a heavy chain variable region with an amino acid sequence of SEQ ID NO: 304 and a light chain variable region with an amino acid sequence of SEQ ID NO: 328 as disclosed herein, (21) a heavy chain variable region with an amino acid sequence of SEQ ID NO: 304 and a light chain variable region with an amino acid sequence of SEQ ID NO: 336 as disclosed herein, (22) a heavy chain variable region with an amino acid sequence of SEQ ID NO: 304 and a light chain variable region with an amino acid sequence of SEQ ID NO: 344 as disclosed herein, (23) a heavy chain variable region with an amino acid sequence of SEQ ID NO: 304 and a light chain variable region with an amino acid sequence of SEQ ID NO: 352 as disclosed herein, (24) a heavy chain variable region with an amino acid sequence of SEQ ID NO: 304 and a light chain variable region with an amino acid sequence of SEQ ID NO: 360 as disclosed herein, (25) a heavy chain variable region with an amino acid sequence of SEQ ID NO: 304 and a light chain variable region with an amino acid sequence of SEQ ID NO: 368 as disclosed herein or (26) a heavy chain variable region with an amino acid sequence of SEQ ID NO: 304 and a light chain variable region with an amino acid sequence of SEQ ID NO: 376 as disclosed herein.

In specific embodiments, the antibodies or antibody fragments of the present invention comprise at least one CDR, wherein the amino acid sequence of the CDR comprises, consists essentially of or consist of an amino acid sequence that is at least 70%, 71%, 72%, 73%, 74% 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% identical to the amino acid sequence of SEQ ID NO: 2, provided the antibodies or antibody fragments are not any of enumerated exceptions 1-26 discussed above. In more specific embodiments, the antibodies or antibody fragments comprise, consist essentially of or consist of at least two CDRs

In particular, the present invention provides antibodies or antibody fragments that bind to the four hydrophobic pockets in the head of the G glycoprotein of the Hendra virus and/or Nipah virus. The antibodies may be monoclonal or polyclonal. The primary amino acid structure and the secondary and tertiary structures of the of the G glycoprotein of the Hendra virus and/or Nipah virus are well known. Hendra virus and Nipah virus, in general, begin the infection process by binding to the ephrin B2 transmembrane protein that is present on at least endothelial cells, among others. Specifically, the ephrin B2 protein contains a “GH-loop region” that inserts into the 4 hydrophobic binding pockets on the head of the G glycoprotein of Hendra virus and/or Nipah virus, thus allowing the viruses to bind specifically to the cell surface protein and begin the infection process. The contact residues of Nipah virus that bind the ephrin B2 are V507, F458 and I401, whereas the contact residues of Hendra virus that bind to ephrin B2 are T507, Y458 and V401, with the letters referring to the standard one-letter abbreviation of standard amino acids and the numbering referring to the amino acid numbering according to the UniProt Database Accession Number 089343 (Hendra virus) (SEQ ID N0:30) and Q9IH62 (Nipah virus) (SEQ ID NO:31). As such, the present invention provides antibodies or antibody fragments that bind the non-linear epitope of Nipah virus defined by V507/F458/I401 and/or bind the non-linear epitope of Hendra virus defined by T507/Y458/V401, provided the antibodies or antibody fragments are not any of enumerated exceptions 1-26 discussed above.

A polypeptide having an amino acid sequence at least, for example, about 95% “identical” to a reference amino acid sequence, e.g., SEQ ID NO: 2, is understood to mean that the amino acid sequence of the polypeptide is identical to the reference sequence except that the amino acid sequence may include up to about five modifications per each 100 amino acids of the reference amino acid sequence. In other words, to obtain a peptide having an amino acid sequence at least about 95% identical to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using well known techniques. While there are several methods to measure identity between two polynucleotide or polypeptide sequences, the term “identity” is well known to skilled artisans (Carillo (1988) J. Applied Math. 48, 1073). Examples of computer program methods to determine identity and similarity between two sequences include, but are not limited to, GCG program package (Devereux (1984) Nucleic Acids Research 12, 387), BLASTP, ExPASy, BLASTN, FASTA (Atschul (1990) J. Mol. Biol. 215, 403) and FASTDB. Examples of methods to determine identity and similarity are discussed in Michaels (2011) Current Protocols in Protein Science, Vol. 1, John Wiley & Sons.

In one embodiment of the present invention, the algorithm used to determine identity between two or more polypeptides is BLASTP. In another embodiment of the present invention, the algorithm used to determine identity between two or more polypeptides is FASTDB, which is based upon the algorithm of Brutlag (1990) Comp. App. Biosci. 6, 237-245). In a FASTDB sequence alignment, the query and reference sequences are amino sequences. The result of sequence alignment is in percent identity. In one embodiment, parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent identity. For query sequences truncated at the N- or C-termini, relative to the reference sequence, the percent identity is corrected by calculating the number of residues of the query sequence that are N- and C-terminus to the reference sequence that are not matched/aligned, as a percent of the total bases of the query sequence. The results of the FASTDB sequence alignment determine matching/alignment. The alignment percentage is then subtracted from the percent identity, calculated by the above FASTDB program using the specified parameters, to arrive at a final percent identity score. This corrected score can be used for the purposes of determining how alignments “correspond” to each other, as well as percentage identity. Residues of the reference sequence that extend past the N- or C-termini of the query sequence may be considered for the purposes of manually adjusting the percent identity score. That is, residues that are not matched/aligned with the N- or C-termini of the comparison sequence may be counted when manually adjusting the percent identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent identity score calculated by the FASTDB program. If the remaining 90 residues were perfectly matched (100% alignment) the final percent identity would be 90% (100% alignment −10% unmatched overhang). In another example, a 90 residue query sequence is compared with a 100 reference sequence, except that the deletions are internal deletions. In this case the percent identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent identity. The addition in the query sequence occurs at the N-terminus of the query sequence and therefore, the FASTDB alignment may not show a match/alignment of the first 10 residues at the N-terminus. If the remaining 100 amino acid residues of the query sequence have 95% identity to the entire length of the reference sequence, the N-terminal addition of the query would be ignored and the percent identity of the query to the reference sequence would be 95%.

As used herein, the terms “correspond(s) to” and “corresponding to,” as they relate to sequence alignment, are intended to mean enumerated positions within the reference protein and those positions in the modified peptide that align with the positions on the reference protein. Thus, when the amino acid sequence of a subject or query peptide is aligned with the amino acid sequence of a reference peptide, e.g., SEQ ID NO: 2, the amino acids in the subject sequence that “correspond to” certain enumerated positions of the reference sequence are those that align with these positions of the reference sequence, e.g., SEQ ID NO: 2, but are not necessarily in these exact numerical positions of the reference sequence. Methods for aligning sequences for determining corresponding amino acids between sequences are described herein. Accordingly, the invention provides novel peptides whose sequences correspond to the sequence of SEQ ID NO: 2.

Variants resulting from insertion of a polynucleotide encoding the novel peptides into an expression vector system are also contemplated. For example, variants (usually insertions) may arise from when the amino terminus and/or the carboxy terminus of a novel peptide is/are fused to another polypeptide.

In another aspect, the invention provides deletion variants wherein one or more amino acid residues in the novel peptides are removed. Deletions can be effected at one or both termini of the peptides, or with removal of one or more non-terminal amino acid residues.

Within the confines of the disclosed percent identities, the invention also relates to substitution variants of disclosed peptides of the invention. Substitution variants include those polypeptides wherein one or more amino acid residues of an amino acid sequence are removed and replaced with alternative residues. In one aspect, the substitutions are conservative in nature; however, the invention embraces substitutions that are also non-conservative. Conservative substitutions for the purposes of the present invention may be defined as set out in the tables below. Amino acids can be classified according to physical properties and contribution to secondary and tertiary protein structure. A conservative substitution is recognized in the art as a substitution of one amino acid for another amino acid that has similar properties. Exemplary conservative substitutions are set out in below.

TABLE III
Conservative Substitutions

| Side Chain Characteristic | Amino Acid |
|---|---|
| Aliphatic | |
| Non-polar | Gly, Ala, Pro, Iso, Leu, Val |
| Polar-uncharged | Cys, Ser, Thr, Met, Asn, Gln |
| Polar-charged | Asp, Glu, Lys, Arg |
| Aromatic | His, Phe, Trp, Tyr |
| Other | Asn, Gln, Asp, Glu |

Alternatively, conservative amino acids can be grouped as described in Lehninger (1975) Biochemistry, Second Edition; Worth Publishers, pp. 71-77, as set forth below.

TABLE IV
Conservative Substitutions

| Side Chain Characteristic | Amino Acid |
|---|---|
| Non-polar (hydrophobic) | |
| Aliphatic: | Ala, Leu, Iso, Val, Pro |
| Aromatic: | Phe, Trp |
| Sulfur-containing: | Met |
| Borderline: | Gly |
| Uncharged-polar | |
| Hydroxyl: | Ser, Thr, Tyr |
| Amides: | Asn, Gln |
| Sulfhydryl: | Cys |
| Borderline: | Gly |
| Positively Charged (Basic): | Lys, Arg, His |
| Negatively Charged (Acidic) | Asp, Glu |

And still other alternative, exemplary conservative substitutions are set out below.

TABLE V
Conservative Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human framing regions (FRs) and/or Fc/pFc′ regions to produce a functional antibody or antibody fragment. For example, PCT International Publication Number WO 92/04381 teaches the production and use of humanized murine RSV antibodies in which at least a portion of the murine FR regions have been replaced by FR regions of human origin. It is also possible, in accordance with the present invention, to produce chimeric antibodies including non-human sequences. For example, murine, ovine, equine, bovine, non-human primate or other mammalian Fc or FR sequences can be used to replace some or all of the Fc or FR regions of Hendra and Nipah antibodies.

The present invention also provides for F(ab′)2, Fab, Fv and Fd fragments of Hendra and Nipah antibodies, as well as chimeric antibodies or antibody fragments in which the Fc and/or FR and/or, CDR1 and/or CDR2 and/or CDR3 light chain or heavy chain regions of the Hendra and Nipah monoclonal have been replaced by homologous human or non-human sequences. For example, the invention provides chimeric Fab and/or F(ab′)2 fragments in which the FR and/or CDR1 and/or CDR2 and/or CDR3 light chain or heavy chain regions of the Hendra and Nipah antibodies have been replaced by homologous human or non-human sequences. The invention also provides for chimeric Fd fragment antibodies in which the FR and/or CDR1 and/or CDR2 and/or CDR3 heavy chain regions have been replaced by homologous human or non-human sequences. Such CDR grafted or chimeric antibodies or antibody fragments can be effective in prevention and treatment of Hendra or Nipah virus infection.

In select embodiments, the chimeric antibodies or antibody fragments of the invention are fully human monoclonal antibodies including at least the novel peptides of the present invention, which can be used as heavy chain CDR3 regions in the antibodies or antibody fragments. As noted above, such chimeric antibodies may be produced in which some or all of the FR regions of the Hendra and Nipah antibodies or antibody fragments have been replaced by other homologous human FR regions. In addition, the Fc portions may be replaced so as to produce IgA or IgM as well as IgG antibodies bearing some or all of the CDRs of the Hendra and Nipah antibodies or antibody fragments. In select embodiments, administration of the antibodies, antibody fragments, chimeric antibodies or chimeric antibody fragments will not evoke an immune response.

It is possible to determine, without undue experimentation, if any of the antibodies or antibody fragments described herein have specificity towards at least a portion of the Hendra and/or Nipah viruses using standard techniques well known to one of skill in the art. For example, the antibody or antibody fragment can be tested for its ability to can compete with known Hendra or Nipah antibodies to bind to Hendra or Nipah virus, e.g., as demonstrated by a decrease in binding of the known Hendra or Nipah antibodies. Screening of Hendra and/or Nipah antibodies or antibody fragments can also be carried out by utilizing Hendra and/or Nipah viruses and determining whether the test antibodies or antibody fragments neutralize the virus.

By using the antibodies or antibody fragments of the invention, it is also possible to produce anti-idiotypic antibodies which can be used to screen other antibodies to identify whether the antibody has the same binding specificity as an antibody of the invention. In addition, such anti-idiotypic antibodies can be used for active immunization (Herlyn, D. et al. 1986 Science 232:100-102). Such anti-idiotypic antibodies can be produced using well-known hybridoma techniques (Kohler, G. and Milstein, C. 1975 Nature 256:495-497). An anti-idiotypic antibody is an antibody which recognizes unique determinants present on an antibody produced by the cell line of interest. These determinants are located in the hypervariable region of the antibody. It is this region which binds to a given epitope and, thus, is responsible for the specificity of the antibody. An anti-idiotypic antibody can be prepared by immunizing an animal with the monoclonal antibody of interest. The immunized animal will recognize and respond to the idiotypic determinants of the immunizing antibody and produce an antibody to these idiotypic determinants. By using the anti-idiotypic antibodies of the immunized animal, which are specific for the monoclonal antibodies of the invention, it is possible to identify other clones with the same idiotype as the antibody of the hybridoma used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

The present invention also provides nucleic acids encoding the novel peptides of the present invention as well as proteins and peptides comprising the novel peptides of the present invention. Such nucleic acids may or may not be operably joined to other nucleic acids forming a recombinant vector for cloning or for expression of the peptides of the present invention. The present invention thus includes any recombinant vector containing coding sequences of the novel peptides of the present invention, or part thereof, whether for prokaryotic or eukaryotic transformation, transfection or gene therapy. Such vectors may be prepared using conventional molecular biology techniques, known to those with skill in the art. Recombinant techniques would include but are not limited to utilizing DNA coding sequences for the immunoglobulin V-regions of the Hendra and Nipah antibodies or antibody fragments, including framework and CDRs or parts thereof, and a suitable promoter either with (Whittle, N. et al. 1987 Protein Eng 1:499-505 and Burton, D. R. et al. 1994 Science 266:1024-1027) or without (Marasco, W. A. et al. 1993. Proc Natl Acad Sci USA 90:7889-7893 and Duan, L. et al. 1994 Proc Natl Acad Sci USA 91:5075-5079) a signal sequence for export or secretion. Such vectors may be transformed or transfected into prokaryotic (Huse, W. D. et al. 1989 Science 246:1275-1281; Ward, S. et al. 1989 Nature 341:544-546; Marks, J. D. et al. 1991 J Mol Biol 222:581-597; and Barbas, C. F. et al. 1991 Proc Natl Acad Sci USA 88:7978-7982) or eukaryotic (Whittle, N. et al. 1987 Protein Eng 1:499-505 and Burton, D. R. et al. 1994 Science 266:1024-1027) cells or used for gene therapy (Marasco, W. A. et al. 1993 Proc Natl Acad Sci USA 90:7889-7893 and Duan, L. et al. 1994 Proc Natl Acad Sci USA 91:5075-5079) by conventional techniques, known to those with skill in the art.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells which have been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques. Some vectors that may be utilized include but are not limited to vectors that are capable of autonomous replication and expression of the structural gene products present in the DNA segments to which they are operably joined.

As used herein, a coding sequence and regulatory sequences are said to be “operably joined” or “operably connected” when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but in general include but are not limited to 5′ non-transcribing and 5′ non-translating sequences involved with initiation of transcription and translation respectively, such as a TATA box, capping sequence, CAAT sequence, and the like. In particular, a 5′ non-transcribing regulatory sequence may include a promoter region which includes a promoter sequence for transcriptional control of the operably joined coding sequence. Regulatory sequences may also include enhancer sequences or upstream activator sequences, as desired.

The vectors of the present invention may or may not be expression vectors. Expression vectors include regulatory sequences operably joined to a nucleotide sequence encoding one of the novel peptides, antibodies or antibody fragments of the invention. As used herein, the term “regulatory sequences” means nucleotide sequences necessary for or conducive to the transcription of a nucleotide sequence encoding a desired peptide and/or which are necessary for or conducive to the translation of the resulting transcript into the desired peptide. Regulatory sequences include, but are not limited to, 5′ sequences such as operators, promoters and ribosome binding sequences, and 3′ sequences such as polyadenylation signals. The vectors of the invention may optionally include 5′ leader or signal sequences, 5′ or 3′ sequences encoding fusion products to aid in protein purification, and various markers which aid in the identification or selection of transformants. The choice and design of an appropriate vector is within the ability and discretion of one of ordinary skill in the art. The subsequent purification of the antibodies may be accomplished by any of a variety of standard means known in the art.

The present invention also provides for host cells, both prokaryotic and eukaryotic comprising at least one nucleic acid encoding the novel peptides of the present invention, including but not limited to the vectors of the present invention.

In one embodiment using a prokaryotic expression host, the vector utilized includes a prokaryotic origin of replication or replicon, i.e., a DNA sequence having the ability to direct autonomous replication and maintenance of the recombinant DNA molecule extrachromosomally in a prokaryotic host cell, such as a bacterial host cell, transformed therewith. Such origins of replication are well known in the art.

One method of achieving high levels of gene expression in E. coli includes but is not limited to the use of strong promoters to generate large quantities of mRNA and also ribosome binding sites to ensure that the mRNA is efficiently translated. For example, ribosome binding sites in E. coli include an initiation codon (AUG) and a sequence 3-9 nucleotides long located 3-11 nucleotides upstream from the initiation codon (Shine J. and Dalgamo L. 1975 Nature 254:34-38). The sequence, which is called the Shine-Dalgarno (SD) sequence, is complementary to the 3′ end of E. coli 16S rRNA. Binding of the ribosome to mRNA and the sequence at the 3′ end of the mRNA can be affected by several factors: the degree of complementarity between the SD sequence and 3′ end of the 16S rRNA, the spacing lying between the SD sequence and the AUG and even the nucleotide sequence following the AUG, which affects ribosome binding. The 3′ regulatory sequences may or may not define at least one termination (stop) codon in frame with and operably joined to the heterologous fusion polypeptide.

In addition, those embodiments that include a prokaryotic replicon may or may not include a gene whose expression confers a selective advantage, such as drug resistance, to a bacterial host transformed therewith. Typical bacterial drug resistance genes are those that confer resistance to ampicillin, tetracycline, neomycin/kanamycin or chloramphenicol. Vectors typically also contain convenient restriction sites for insertion of translatable DNA sequences. Exemplary vectors are the plasmids pUC18 and pUC19 and derived vectors such as those that are commercially available.

The antibodies or antibody fragments of the present invention may additionally, of course, be produced by eukaryotic cells such as CHO cells, human or mouse hybridomas, immortalized B-lymphoblastoid cells, and the like. In this case, a vector is constructed in which eukaryotic regulatory sequences are operably joined to the nucleotide sequences encoding one or more peptides of the present invention. The design and selection of an appropriate eukaryotic vector is within the ability and discretion of one of ordinary skill in the art. The subsequent purification of the antibodies may be accomplished by any of a variety of standard means known in the art.

The antibodies or antibody fragments of the present invention may furthermore, of course, be produced in plants. In 1989, Hiatt A. et al. (Nature 342:76-78 (1989)) first demonstrated that functional antibodies could be produced in transgenic plants. Since then, a considerable amount of effort has been invested in developing plants for antibody (or “plantibody”) production (for reviews see Giddings, G. et al., Nat. Biotechnol., 18:1151-1155 (2000); Fischer, R. and Emans, N., Transgenic Res., 9:279-299 (2000)).

One vector useful for screening monoclonal antibodies is a recombinant DNA molecule containing a nucleotide sequence that codes for and is capable of expressing a fusion polypeptide containing, in the direction of amino- to carboxy-terminus, (1) a prokaryotic secretion signal domain, (2) a peptide of the invention, and, optionally, (3) a fusion protein domain. The vector includes DNA regulatory sequences for expressing the fusion polypeptide, for example prokaryotic regulatory sequences. Such vectors can be constructed by those of ordinary skill in the art and have been described by Smith, G. P. et al. (Science 228:1315-1317 (1985)); Clackson, T. et al. (Nature 352:624-628 (1991)); Kang et al. (Methods: A Companion to Methods in Enzymology, vol. 2, R. A. Lerner and D. R. Burton, ed. Academic Press, NY, pp 111-118 (1991)); Batbas, C. F. et al. (Proc Natl Acad Sci USA 88:7978-7982 (1991)); Roberts, B. L. et al. (Proc Natl Acad Sci USA 89:2429-2433 (1992)).

A fusion polypeptide may be useful for purification of the antibodies of the invention. The fusion domain may, for example, include a His tag that allows for purification of the peptide, or a maltose binding protein of the commercially available vector pMAL (New England BioLabs, Beverly, Mass.). A fusion domain that may be useful is a filamentous phage membrane anchor that is particularly useful for screening phage display libraries of monoclonal antibodies.

A secretion signal is a leader peptide domain of a protein that targets the protein to a region, such as the plasma membrane, of the host cell. For example, one secretion signal is the E. coli is a pelB secretion signal. The leader sequence of the pelB protein has previously been used as a secretion signal for fusion proteins (Better, M. et al. Science 240:1041-1043 (1988); Sastry, L. et al. Proc Natl Acad Sci USA 86:5728-5732 (1989); and Mullinax, R. L. et al., Proc Natl Acad Sci USA 87:8095-8099 (1990)). Amino acid residue sequences for other secretion signal polypeptide domains from E. coli useful in this invention can be found in Neidhard, F. C. (ed.), 1987 in Escherichia coli and Salmonella Typhimurium: Typhimurium Cellular and Molecular Biology, American Society for Microbiology, Washington, D.C.

When the antibodies or antibody fragments of the invention include heavy chain and light chain sequences, these sequences may be encoded on separate vectors or, more conveniently, may be expressed by a single vector. The heavy and light chain may, after translation or after secretion, form the heterodimeric structure of natural antibody molecules. Such a heterodimeric antibody may or may not be stabilized by disulfide bonds between the heavy and light chains.

A vector for expression of heterodimeric antibodies, such as full-length antibodies or antibody fragments of the invention, is a recombinant DNA molecule adapted for receiving and expressing translatable first and second DNA sequences. That is, a DNA expression vector for expressing a heterodimeric antibody or antibody fragment provides a system for independently cloning (inserting) two or more translatable DNA sequences into two or more separate cassettes present in the vector, to form two or more separate cistrons for expressing the first and second polypeptides of a heterodimeric antibody or antibody fragment. The DNA expression vector for expressing two cistrons is referred to as a dicistronic expression vector.

In general, a dicistronic expression vector comprises a first cassette that includes upstream and downstream DNA regulatory sequences operably joined via a sequence of nucleotides adapted for directional ligation to an insert DNA. The upstream translatable sequence may encode the secretion signal as described above. The cassette also may include DNA regulatory sequences for expressing the first peptide that is produced when an insert translatable DNA sequence (insert DNA) is directionally inserted into the cassette via the sequence of nucleotides adapted for directional ligation.

The dicistronic expression vector may also contain a second cassette for expressing the second peptide. The second cassette may also include a second translatable DNA sequence that encodes a secretion signal, as described above, that may be operably joined at its 3′ terminus via a sequence of nucleotides adapted for directional ligation to a downstream DNA sequence of the vector that typically defines at least one stop codon in the reading frame of the cassette. The second translatable DNA sequence can be operably joined at its 5′ terminus to DNA regulatory sequences forming the 5′ elements. Upon insertion of a translatable DNA sequence (insert DNA), the second cassette is capable of expressing the second fusion polypeptide comprising a secretion signal with a polypeptide coded by the insert DNA.

The invention also provides for methods of making any of the novel, inventive peptides of the present invention. In certain embodiments, the methods of making the novel peptides of the present invention include making antibodies or antibody fragments that comprise at least one novel peptide of the present invention. The methods of making the novel peptides, or making antibodies or antibody fragments comprising the novel peptides, include but are not limited to culturing the novel, inventive host cells of the present invention under conditions suitable for protein expression and isolating the peptides from culture. The host cells used in the methods of making peptides of the present invention may or may not include nucleic acids that encode antibodies or antibody fragments comprising the novel peptides of the present invention. The produced peptides or produced antibodies or antibody fragments may or may not be substantially pure.

As used herein with respect to polypeptides, the term “substantially pure” is used to mean that the polypeptides are essentially free of other substances with which they may be found in nature or in vivo systems to an extent practical and appropriate for their intended use. In particular, the polypeptides are sufficiently pure and are sufficiently free from other biological constituents of their host cells so as to be useful in, for example, generating antibodies, sequencing, or producing pharmaceutical preparations. By techniques well known in the art, substantially pure polypeptides may be produced in light of the nucleic acid and amino acid sequences disclosed herein. Because a substantially purified polypeptide of the invention may be admixed with a pharmaceutically acceptable carrier in a pharmaceutical preparation, the polypeptide may comprise only a certain percentage by weight of the preparation. The polypeptide is nonetheless substantially pure in that it has been substantially separated from the substances with which it may be associated in living systems.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

Methods of culturing host cells to produce proteins, including antibodies or antibody fragments comprising the novel peptides of the present invention, are well known in the art and such methods need not be repeated herein. One of skill in the art will readily recognize that the culture conditions necessary for protein production depend upon, among other things, the type of host cell being cultured, the nature of the protein or peptide being produced and the quantity desired.

The invention also provides methods for preparing diagnostic or pharmaceutical compositions comprising the peptides of the present invention, which may or may not be part of an antibody or antibody fragment. The invention also provides methods for preparing diagnostic or pharmaceutical compositions comprising the novel nucleic acid sequences encoding the novel peptides of the invention or part thereof. The pharmaceutical compositions of the present invention can be used for treating symptoms of Hendra Virus Disease or Nipah Virus Disease in a subject in need thereof, or can be used for treating Hendra Virus Disease or Nipah Virus Disease itself in a subject in need thereof.

Accordingly, the present invention provides methods of treating a subject with a Hendra virus or Nipah virus infection comprising administering a therapeutically effective amount of at least one peptide of the present invention to a subject in need thereof. In a more specific embodiment, the invention provides for methods of treating a subject with a Hendra virus or Nipah virus infection comprising administering a therapeutically effective amount at least one antibody or antibody fragment, wherein the antibody or antibody fragment comprises, consists essentially of or consists of at least one novel peptide of the present invention to a subject in need thereof.

As used herein, a “therapeutically effective amount” of the peptides, antibodies or antibody fragments of the invention is a dosage large enough to produce the desired effect in which the symptoms of Hendra Virus Disease or Nipah Virus Disease are ameliorated or the likelihood of infection is decreased. A therapeutically effective amount is generally not a dose so large as to cause adverse side effects, such as but not limited to hyperviscosity syndromes, pulmonary edema, congestive heart failure, and the like. Generally, a therapeutically effective amount may vary with the subject's age, condition, and sex, as well as the extent of the disease in the subject and can be determined by one of skill in the art. The dosage of the therapeutically effective amount may be adjusted by the individual physician or veterinarian in the event of any complication. A therapeutically effective amount may vary from about 0.01 mg/kg to about 50 mg/kg, specifically from about 0.1 mg/kg to about 20 mg/kg, more specifically from about 0.2 mg/kg to about 2 mg/kg. The peptides, antibodies or antibody fragments may be administered once or more than once in a single day or over a period of days.

The present invention also provides prophylactic methods as well. Indeed, the present invention provides methods of preventing or reducing the likelihood of acquiring a Hendra virus or Nipah virus infection and preventing or reducing the likelihood of acquiring a disease or condition associated with Hendra viruses or Nipah virus infection. The prevention methods comprise administering a prophylactically effective amount of at least one peptide of the present invention to a subject. In a more specific embodiment, the invention provides for methods of reducing the likelihood of acquiring a condition or disease associated with Hendra virus or Nipah virus infection comprising administering a prophylactically effective amount of at least one antibody or antibody fragment, wherein the antibody or antibody fragment comprises, consists essentially of or consists of at least one novel peptide of the present invention to a subject. The subject on which the prevention or prophylactic methods are practiced may or may not be a higher risk of acquiring a condition or disease associated with Hendra virus or Nipah virus infection than another subject from a different population.

As used herein, a “prophylactically effective amount” of the peptides, antibodies or antibody fragments of the invention is a dosage large enough to produce the desired effect in the protection of individuals against Hendra or Nipah virus infection for a reasonable period of time, such as one to two months or longer following administration. Generally, a prophylactically effective amount may vary with the subject's age, condition, and sex, as well as the extent of the disease in the subject and can be determined by one of skill in the art. The dosage of the prophylactically effective amount may be adjusted by the individual physician or veterinarian in the event of any complication. A prophylactically effective amount may vary from about 0.01 mg/kg to about 50 mg/kg, specifically from about 0.1 mg/kg to about 20 mg/kg, more specifically from about 0.2 mg/kg to about 2 mg/kg, in one or more administrations (priming and boosting).

The treatment and prevention methods herein may or may not include screening a subject to determine if the subject has been infected with Hendra virus and/or Nipah virus or is at risk of being infected with Hendra virus or Nipah virus.

As used herein, “administer” or variations thereof is used to mean bringing the one or more novel peptides into proximity with a cell or group of cells, including cells comprised within a living, whole organism, such that the one or more novel peptides can exert a biological effect on the cells. Of course, “administering” the novel peptides of the present invention can be achieved by administering an antibody or antibody fragment comprising one or more novel peptides to a subject in need thereof. Thus, in one embodiment of the present invention, “administer” can mean a stable or transient transfection of DNA or RNA molecule(s) into cells, where the cells may or may not be part of a living, whole organism. In another embodiment, the peptides or antibodies or antibody fragments comprising the novel peptides can be administered repeatedly to the subject.

As used herein, the terms “Hendra Virus Disease” and “Nipah Virus Disease” refer to diseases caused, directly or indirectly, by infection from Hendra or Nipah virus. The broad species tropisms and the ability to cause fatal disease in both animals and humans have distinguished Hendra virus (HeV) and Nipah virus (NiV) from all other known paramyxoviruses (Eaton B. T. Microbes Infect 3:277-278 (2001)). These viruses can be amplified and cause disease in large animals and can be transmitted to humans where infection is manifested as a severe respiratory illness and/or febrile encephalitis.

The pharmaceutical preparation includes a pharmaceutically acceptable carrier. Such carriers, as used herein, means a material that does not interfere with the effectiveness of the biological activity of the active ingredients. The term “physiologically acceptable” refers to a material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The peptides, antibodies or antibody fragments of the invention can be administered by injection or by gradual infusion over time. The administration of the peptides, antibodies or antibody fragments of the invention may, for example, be intravenous, intraperitoneal, intramuscular, intracavity, subcutaneous, or transdermal. Techniques for preparing injectate or infusate delivery systems containing antibodies are well known to those of skill in the art. Generally, such systems should utilize components which will not significantly impair the biological properties of the peptides, antibodies or antibody fragments such as the paratope binding capacity (see, for example, Remington's Pharmaceutical Sciences, 18th edition, 1990, Mack Publishing). Those of skill in the art can readily determine the various parameters and conditions for producing injectates or infusates without resort to undue experimentation.

For example, preparations for parenteral administration include sterile aqueous or non-aqueous solutions, suspensions, and emulsions. Examples of non-aqueous solvents include but are not limited to propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Parenteral vehicles include but are not limited to sodium chloride solution, Ringer's dextrose, dextrose and sodium chloride, lactated Ringer's or fixed oils. Intravenous vehicles include but are not limited to fluid and nutrient replenishers, electrolyte replenishers (such as those based on Ringer's dextrose), and the like. Preservatives and other additives may also be present such as, for example, antimicrobials, anti-oxidants, chelating agents, and the like.

The peptides, antibodies or antibody fragments of the invention are suited for in vitro use, for example, in immunoassays in which they can be utilized in liquid phase or bound to a solid phase carrier. In addition, the peptides, antibodies or antibody fragments in these immunoassays can be detectably labeled in various ways. Examples of types of immunoassays which can utilize the peptides, antibodies or antibody fragments of the invention are competitive and non-competitive immunoassays in either a direct or indirect format. Examples of such immunoassays are the radioimmunoassay (RIA) and the sandwich (immunometric) assay. Detection of antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The anti-Hendra and anti-Nipah peptides, antibodies or antibody fragments of the invention may be labeled by a variety of means for use in diagnostic and/or pharmaceutical applications. There are many different labels and methods of labeling known to those of ordinary skill in the art. Examples of the types of labels which can be used in the present invention include but are not limited to enzymes, radioisotopes, fluorescent compounds, colloidal metals, chemiluminescent compounds and bioluminescent compounds. One of ordinary skill in the art will readily be able to determine suitable labels for binding to the peptides, antibodies or antibody fragments of the invention. Furthermore, the binding of these labels to the peptides, antibodies or antibody fragments of the invention can be done using standard techniques common to those of ordinary skill in the art.

Another labeling technique which may result in greater sensitivity consists of coupling the peptides, antibodies or antibody fragments to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

The peptides, antibodies or antibody fragments of the invention can be bound to many different carriers and used to detect the presence of Hendra or Nipah virus. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble for purposes of the invention. Those skilled in the art will know of other suitable carriers for binding peptides, antibodies or antibody fragments, or will be able to ascertain such, using routine experimentation.

For purposes of the invention, Hendra or Nipah virus may be detected by the peptides, antibodies or antibody fragments of the invention when present in biological fluids and tissues. Any sample containing a detectable amount of Hendra or Nipah virus can be used. A sample can be a liquid such as urine, saliva, cerebrospinal fluid, blood, serum or the like; a solid or semi-solid such as tissues, feces, or the like; or, alternatively, a solid tissue such as those commonly used in histological diagnosis.

The invention also provides for methods of diagnosis and in vivo detection of Hendra virus and Nipah virus using the peptides, antibodies or antibody fragments of the present invention. In using the peptides, antibodies or antibody fragments of the invention for the in vivo detection of antigen, the detectably labeled peptides, antibodies or antibody fragments are given in a dose which is diagnostically effective. The term “diagnostically effective” means that the amount of detectably labeled peptides, antibodies or antibody fragments are administered in sufficient quantity to enable detection of the site having the Hendra or Nipah virus antigen for which the peptides, antibodies or antibody fragments are specific.

The concentration of detectably labeled peptide, antibody or antibody fragment which is administered should be sufficient such that the binding to Hendra or Nipah virus is detectable compared to the background.

As a rule, the dosage of detectably labeled peptides, antibodies or antibody fragments for in vivo diagnosis will vary depending on such factors as age, sex, and extent of disease of the individual. The dosage of peptides, antibodies or antibody fragments can vary from about 0.01 mg/kg to about 50 mg/kg, specifically from about 0.1 mg/kg to about 20 mg/kg, more specifically from about 0.1 mg/kg to about 2 mg/kg. Such dosages may vary, for example, depending on whether multiple injections are given, on the tissue being assayed, and other factors known to those of skill in the art.

For in vivo diagnostic imaging, the type of detection instrument available is a one factor in selecting an appropriate label, such as but not limited to a radioisotope. For example, the radioisotope chosen must have a type of decay which is detectable for the given type of instrument. Still another factor in selecting an appropriate label for in vivo diagnosis is that the half-life of the label must be long enough such that it is still detectable at the time of maximum uptake by the target, but short enough such that any deleterious effect to the host is acceptable.

For in vivo diagnosis, the label(s) may be bound to the peptides, antibodies or antibody fragments of the invention either directly or indirectly by using an intermediate functional group. Intermediate functional groups which often are used to bind labels, such as for example radioisotopes, can exist as metallic ions and may be bifunctional chelating agents such as diethylenetriaminepentacetic acid (DTPA) and ethylenediaminetetra-acetic acid (EDTA) and similar molecules. Typical examples of metallic ions which can be bound to the peptides, antibodies or antibody fragments of the invention are ¹¹¹In, ⁹⁷Ru, ⁶⁷Ga, ⁶⁸Ga, ⁷²As, ⁸⁹Zr and ²⁰¹Tl to name a few.

The peptides, antibodies or antibody fragments of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include but are not limited to ¹⁵⁷Gd, ⁵⁵Mn, ¹⁶²Dy, ⁵²Cr and ⁵⁶Fe.

The peptides, antibodies or antibody fragments of the invention can be used in vitro and in vivo to monitor the course of Hendra Virus Disease or Nipah Virus Disease therapy. Thus, for example, by measuring the increase or decrease in the number of cells infected with Hendra or Nipah virus over time, i.e., measuring at a first and second time point, or changes in the concentration of Hendra or Nipah virus present in the body or in various body fluids over time, it would be possible to determine whether a particular therapeutic regimen aimed at ameliorating Hendra Virus Disease or Nipah Virus Disease is effective.

The materials for use in the diagnostic assays that the invention provides are ideally suited for the preparation of a kit. Such a kit may comprise a carrier that is compartmentalized to receive in close confinement one or more containers such as vials, tubes, and the like, with each of the container comprising one of the separate elements to be used in the method. For example, one of the containers may comprise a peptide, antibody or antibody fragment of the invention that is, or can be, detectably labeled. The kit may also have containers containing buffer(s) and/or a container comprising a reporter, such as but not limited to a biotin-binding protein, such as avidin or streptavidin, bound to a reporter molecule, such as an enzymatic or fluorescent label.

Measuring the ability of the peptides, antibodies or antibody fragments of the present invention to inhibit fusion mediated by HeV envelope glycoprotein (Env) expressing cells with cells that we had previously identified as fusion-competent can be used to test the neutralizing activity of the peptides, antibodies or antibody fragments of the present invention. Fusion can be measured by two assays—a reporter gene assay and a syncytia formation assay. Methods of measuring fusion of the virus are reported in U.S. Pat. No. 7,988,971, which is incorporated by reference in its entirety.

Neutralization assays utilizing infectious HeV and NiV can also be used to test the inhibitory activity of the peptides, antibodies or antibody fragments. Such neutralization assays are reported in U.S. Pat. No. 7,988,971.

The Examples and Figures describe how the antibodies or antibody fragments of the present invention can be assayed and evaluated for antigen binding to both wild-type G protein and possible escape mutants of G protein. Structure-based targeted amino acid mutations may be made and the resulting antibody variants in form of Fab fragments can be expressed and then tested for antigen binding in ELISA using G protein antigen bound to plates. Also, variants made can be used as competitors for blocking the interactions between G protein and its ephrin receptors in a competition-based ELISA assay.

### Example 1

The antibodies or antibody fragments that retain binding and virus neutralizing activity can be examined in vitro and in vivo to explore whether Hendra and Nipah virus can escape, e.g., through mutation, the ability of the antibodies or antibody fragments to neutralize the virus.

Candidate antibodies or antibody fragments can be tested for therapeutic activity by producing said antibody or fragment thereof either, for example as Fab or IgG format and used to passively immunize animals challenged with Nipah or Hendra virus. For example, virus challenged monkeys are treated with 15 mg/kg by i.v. administration on days 1 and 3 or, days 3 and 5, or days 5 and 7, with two doses total. Control animals are not treated and typically die within 8 to 10 days post challenge. All animals treated with antibodies or antibody fragments that are effective will display a longer survival period after infection by either Hendra or Nipah virus.

### Example 2

Candidate antibodies or antibody fragments can also be examined for whether virus can escape neutralization by serial passing and evaluated by how readily virus can escape, and if possible escape virus is isolated it can be characterized to examine whether it (the escape variant) is weakened or less fit.

Neutralization resistant NiV and HeV mutants can be generated by incubating 1×10⁵ TCID₅₀ of each virus (either Nipah or Hendra) with 100 μg or 10 μg of antibodies or antibody fragments of the present invention in 100 μl media for 1 h at 37° C. Vero E6 cells (^{˜}10⁶) are then inoculated with the “pre-incubated virus” in the presence of the antibodies or antibody fragments at about the same concentration. The development of cytopathic effect (CPE) are monitored over 72 h and progeny viruses harvested. Antibodies or antibody fragment treatment is repeated two additional times with CPE development monitored with each passage. Passage 3 viruses are plaque purified in the presence of mAbs and neutralization resistant viruses would be isolated. Experiments are performed in duplicate and the G glycoprotein genes of individual plaques from each experiment are sequenced to identify escape mutations.

The neutralization titers between wild type and the neutralization resistant virus are also determined by micro-neutralization assay. Briefly, antibodies or antibody fragments are serially diluted two-fold, and incubated with 100 TCID₅₀ of the wild type (WT) virus and neutralization resistant isolates for 1 hour at 37° C. Virus and antibodies are then added to a 96-well plate with about 2×10⁴ Vero E6 cells/well in 4 wells per antibody/fragment dilution. Wells are checked for CPE at 3 days post infection and the 50% neutralization titer is determined as the antibody or antibody fragment concentration at which at least 50% of wells showed no CPE. Once analyzed, the candidate antibodies or antibody fragments are examined for growth characteristics as a measure of viral fitness.

### Example 3

Growth curves are performed by inoculating cell cultures with Nipah or Hendra viruses and their escape mutant clones at a multiplicity of infection (MOI) of 1 for 1 h, after which the cells are washed 3 times with PBS and overlaid with medium. Virus samples are obtained at various time points after infection and stored at −80° C. until viral titers are determined by TCID₅₀. These experiments show how difficult it would be for Nipah and/or Hendra virus to escape from the antibodies or antibody fragments of the present invention. The best candidates that both neutralize virus and to which the virus exhibits poor escapability are produced and prepared as a passive immunotherapeutic to treat a subject exposed to or infected with Nipah virus or Hendra virus.

What is claimed is:

1. A peptide selected from the group consisting of:
a) a peptide comprising an amino acid sequence at least 78% identical to the amino acid sequence of SEQ ID NO: 2,
b) a peptide comprising an amino acid sequence at least 82% identical to the amino acid sequence of SEQ ID NO: 2,
c) a peptide comprising an amino acid sequence at least 86% identical to the amino acid sequence of SEQ ID NO: 2,
d) a peptide comprising an amino acid sequence at least 91% identical to the amino acid sequence of SEQ ID NO: 2,
e) a peptide comprising an amino acid sequence at least 95% identical to the amino acid sequence of SEQ ID NO: 2, and
f) a peptide comprising an amino acid sequence that is 100% identical to the amino acid sequence of SEQ ID NO: 2,
wherein the peptide does not comprise the amino acid sequence of SEQ ID NO: 1.

2. The peptide of claim 1, wherein the peptide comprises an amino acid sequence selected from the group consisting of the amino acid sequence of SEQ ID NO: 3, the amino acid sequence of SEQ ID NO: 4, the amino acid sequence of SEQ ID NO: 5, the amino acid sequence of SEQ ID NO: 6, the amino acid sequence of SEQ ID NO: 7, the amino acid sequence of SEQ ID NO: 8, the amino acid sequence of SEQ ID NO: 9, the amino acid sequence of SEQ ID NO: 10, the amino acid sequence of SEQ ID NO: 11, the amino acid sequence of SEQ ID NO: 12, the amino acid sequence of SEQ ID NO: 13, the amino acid sequence of SEQ ID NO: 14, the amino acid sequence of SEQ ID NO: 15, the amino acid sequence of SEQ ID NO: 16, the amino acid sequence of SEQ ID NO: 17, the amino acid sequence of SEQ ID NO: 18, the amino acid sequence of SEQ ID NO: 19, the amino acid sequence of SEQ ID NO: 20, the amino acid sequence of SEQ ID NO: 21, the amino acid sequence of SEQ ID NO: 22, and the amino acid sequence of SEQ ID NO: 23.

3. An antibody or antibody fragment comprising the peptide of claim 1, wherein the peptide is a heavy chain complementarity determining region (CDR).

4. The antibody or antibody fragment of claim 3, further comprising at least one additional heavy chain CDR.

5. The antibody or antibody fragment of claim 4, wherein the at least one additional heavy chain CDR comprises the amino acid sequence of SEQ ID NO: 25.

6. The antibody or antibody fragment of claim 5, further comprising a second additional heavy chain CDRs.

7. The antibody or antibody fragment of claim 6, wherein the second additional heavy chain CDRs comprises the amino acid sequence of SEQ ID NO: 26.

8. The antibody or antibody fragment of any of claims 3-7, further comprising at least one light chain CDR.

9. The antibody or antibody fragment of claim 8, wherein the at least one light chain CDR comprises the amino acid sequence of SEQ ID NO: 27.

10. The antibody or antibody fragment of claim 9, further comprising a second light chain CDR.

11. The antibody or antibody fragment of claim 10, wherein the second light chain CDR comprises the amino acid sequence of SEQ ID NO: 28.

12. The antibody or antibody fragment of claim 11, further comprising a third light chain CDR.

13. The antibody or antibody fragment of claim 12, wherein the third light chain CDR comprises the amino acid sequence of SEQ ID NO: 29.

14. A method of treating a Hendra virus or Nipah virus infection comprising administering the antibody or antibody fragment of claim 3 to a subject which has been infected with Hendra or Nipah virus.

15. A method of reducing the likelihood of a subject developing a disease caused by Hendra virus or Nipah virus, the method comprising administering the antibody or antibody fragment claim 3 to a subject prior to Hendra virus infection or Nipah virus infection.

16. A nucleic acid encoding the peptide of claim 1.

17. A vector comprising the nucleic acid of claim 16.

18. A host cell comprising the vector of claim 17.

19. A method of making a peptide comprising an amino acid of SEQ ID NO: 2, SEQ ID NO: 3; SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23, the method comprising culturing the host cell of claim 18 under conditions suitable for protein expression and isolating the peptide.

20. An antibody that binds to the four hydrophobic pockets of the G glycoprotein head of Hendra virus or Nipah virus.
